(19) United States
(12) Patent Application Publication
(10) Pub. No.: US 2014/0127269 A1
(43) Pub. Date: May 8, 2014

(54) Anti-Inflammatory Peptide Derived From Thrombospondin-1 and Uses Thereof

(71) Applicant: THE SCHEPENS EYE RESEARCH INSTITUTE, INC., (US)

(72) Inventor: Sharmila Masli, Brookline, MA (US)

(73) Assignee: THE SCHEPENS EYE RESEARCH INSTITUTE, INC., Boston, MA (US)

(21) Appl. No.: 13/766,606

(22) Filed: Feb. 13, 2013

Related U.S. Application Data
(60) Provisional application No. 61/598,201, filed on Feb. 13, 2012.

(51) Int. Cl. A61K 9/00; C07K 7/06

(52) U.S. Cl. CPC A61K 9/0051; C07K 7/06
USPC 424/400; 514/16.5

(57) ABSTRACT

The invention provides compositions and methods for utilizing a peptide of thrombospondin-1 as an anti-inflammatory agent.

### RELATED APPLICATIONS

This application claims the benefit of priority under 35 U.S.C. §119(e) to U.S. Provisional Application No. 61/598,201, filed Feb. 13, 2012. This application is incorporated herein by reference in its entirety.

### STATEMENT AS TO FEDERALLY SPONSORED RESEARCH

This invention was funded in part by the U.S. Government under grant number R01 EY015472, awarded by the National Eye Institute. The Government has certain rights in the invention.

### FIELD OF THE INVENTION

This invention relates generally to the field of ophthalmology.

### BACKGROUND OF THE INVENTION

Inflammation of the ocular and adnexal tissues can occur by a variety of mechanisms and is associated, either primarily or secondarily, with a large number of disease conditions. Current treatments for inflammation of these tissues involve the systemic or intraocular administration of antibiotics, steroids, and immune-system inhibitors. The difficulty of using these systemic drugs becomes apparent through damaging long-term side effects in the case of steroids, long-term drug resistance in the case of antibiotics, or insufficient long-term persistence at the target site in the case of signaling inhibitors. Use of intraocular administration although allows drug delivery at the target site; however, the invasive nature of the treatment poses its own limitations. Moreover, the systemic inhibition of signaling within the immune system can have deleterious outcomes for individuals already afflicted with disease, whose susceptibility to additional complications is increased as a result of the systemic use of these treatments. As such, there is a pressing need to develop new strategies to manage and reduce ocular inflammation.

### SUMMARY OF THE INVENTION

The present invention overcomes these obstacles by the administration of a topical composition comprising an effective amount of an agent which binds to a CD47 receptor on T cells, thereby increasing the population of anti-inflammatory regulatory T (T_{reg}) cells, and decreasing the population of pro-inflammatory T helper 17 (Tₕ17) cells. Specifically, the invention is based on the surprising discovery that a C-terminal peptide of thrombospondin-1 (TSP-1; THBS1) locally decreases or prevents inflammation of the ocular and adnexal tissues.

A method of increasing a population of anti-inflammatory regulatory T cells (T_{reg}) and decreasing a population of pro-inflammatory T helper 17 (Tₕ17) cells in an ocular or adnexal tissue in a subject is carried out by topically administering to an ocular or adnexal tissue of a subject a composition comprising an effective amount of an agent which binds to a CD47 receptor on T cells. The subject is preferably a mammal in need of such treatment, e.g., a subject that has been diagnosed with inflammation (e.g., ocular inflammation) or a predisposition thereto. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. Preferably, the mammal is a human.

The agent that binds to a CD47 receptor on T cells comprises a C-terminal peptide of TSP-1 or a fragment thereof. A peptide fragment of almost any length is employed, so long as it exhibits the desired biological activity (i.e., binds to a CD47 receptor on T cells). For example, illustrative peptide segments with total lengths of about 1,170, about 1,100, about 1,000, about 900, about 800, about 700, about 600, about 500, about 400, about 300, about 200, about 100, about 75, about 50, about 25, about 10, or about 5 amino acids in length (including all intermediate lengths) are included in many implementations of this invention. For example, the C-terminal peptide of TSP-1 is about 5, about 6, about 7, about 8, about 9, about 10, about 11, about 12, about 13, about 14, or about 15 amino acids in length. Preferably, the TSP-1 peptide comprises KRFYVVMWKK (SEQ ID NO: 1). Alternatively, CD-47-binding peptides include the following sequences: FIRVVMYEGKK (SEQ ID NO: 4), RFYVVMWK (SEQ ID NO: 5), and/or KRFYVVMWKK (SEQ ID NO: 6).

The population of anti-inflammatory T_{reg} cells that is expanded by the methods described herein comprises CD4⁺CD25⁺FOXP3⁺ T_{reg} cells which produce transforming growth factor beta (TGF-β). The population of pro-inflammatory Tₕ17 cells that is reduced by the methods described herein produces interleukin-17 (IL-17) or interferon-γ (IFN-γ).

The TSP-1 peptide is administered at a dose of between 0.1 μg and 1 gram, e.g., between 1 μg and 1,000,000 μg; between 10 μg and 100,000 μg; between 100 μg and 10,000 μg; or between 1,000 μg and 5,000 μg. Preferably, the TSP-1 peptide is administered at a dose of 10 μg, e.g., 10 μg per day, or a scaled-up amount appropriate for human therapy.

The composition is present in a concentration range of 0.1-10%, with preferred ranges between 1-5% or 2-2.5% (mg/ml). Exemplary liquid formulations for eye drops contain 2-2.5% (mg/ml) of the composition. Preferred formulations are in the form of a solid, a paste, an ointment, a gel, a liquid, an aerosol, a mist, a polymer, a film, an emulsion, or a suspension. The formulations are administered topically, e.g., the composition is delivered to an ocular or adnexal tissue to directly contact that tissue. For example, the composition is incorporated into or coated onto a contact lens, which is applied directly to the ocular or adnexal tissue. The method does not involve systemic administration or substantial dissemination of the composition to non-ocular or non-adnexal tissue. For example, the method does not involve intravascular administration into vessels infiltrating a tumor or tumor metastasis.

The TSP-1 peptide is administered every 96 hours, every 72 hours, every 48 hours, every 24 hours, every 12 hours, every 6 hours, every 3 hours, or every 1 hour. The TSP-1 peptide is administered for a duration of 1 day, 2 days, 3 days, 5 days, 7 days, 10 days, 20 days, 30 days, 60 days, 90 days, 120 days, or 180 days. For example, the TSP peptide is administered topically once per day for 7 days.

In some cases, the composition further comprises a pharmaceutically acceptable carrier, e.g., a compound selected from the group consisting of a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum.

A method for inhibiting or reducing the severity of an inflammatory disorder affecting the ocular and adnexal tissues is carried out by topically administering to an ocular or adnexal tissue of a subject a composition that binds to a CD47 receptor on T cells. In this manner, the population of T_{reg} cells increases and the population of Tₕ17 cells decreases. The composition that binds to a CD47 receptor on T cells comprises a C-terminal peptide of TSP-1 or a fragment thereof. Preferably, the TSP-1 peptide comprises KRFYVVMWKK (SEQ ID NO: 1).

Inflammation is part of the complex biological response of vascular tissues to harmful stimuli, such as pathogens, damaged cells, or irritants. Thus, subjects characterized as at risk of or suffering from an inflammatory disorder affecting the ocular and adnexal tissues include those subjects that have been exposed to such harmful stimuli or are at risk for exposure to such stimuli. The subject is identified as suffering from an ocular inflammatory disorder by detecting a sign or symptom selected from the group consisting of epithelial overexpression of an inflammatory cytokine, vascular hyperplasia or thickening of lid margin, neovascularization of lid margin or corneal periphery, increase of leukocytes at an ocular or adnexal tissue, or overexpression of a matrix metalloprotease at an ocular or adnexal tissue. Ocular surface inflammation is also characterized by changes in mucous (increased during allergy and reduced during dry eye) and tear secretion, compromised corneal epithelial barrier.

The inflammatory disease may be any acute or chronic inflammatory disease. In one aspect, the inflammatory disease is a non-ocular inflammatory disease selected from the group consisting of autoimmune diseases such as psoriasis, rheumatoid arthritis, inflammatory bowel disease, multiple sclerosis, Sjögren's syndrome, vasculitis, ankylosing spondylitis, transplant rejection disease (i.e., allograft rejection). The inflammatory disease is not cancer or a tumor. Accordingly, the method does not involve intravascular administration into vessels infiltrating a tumor or tumor metastasis. Preferably, the inflammatory disease is an ocular inflammatory disease selected from the group consisting of dry eye disease, uveitis, conjunctivitis, and keratitis. The methods and compositions target the Th1 and Th17 subset of inflammatory cells that have been associated with the listed three categories of ocular inflammation.

Also provided herein are compositions comprising a C-terminal peptide of TSP-1 and a pharmaceutically-acceptable carrier. For example, the C-terminal peptide of TSP-1 is about 5, about 6, about 7, about 8, about 9, about 10, about 11, about 12, about 13, about 14, or about 15 amino acids in length. Preferably, the TSP-1 peptide comprises KRFYVVMWKK (SEQ ID NO: 1). Alternatively, CD-47-binding peptides include the following sequences: FIRVVMYEGKK (SEQ ID NO: 4), RFYVVMWK (SEQ ID NO: 5), and/or KRFYVVMWKK (SEQ ID NO: 6).

Suitable pharmaceutically-acceptable carriers or excipients include a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, detran, gelatin glycerin, polyethylene glycol, poloxamer 407, polysorbate 80, propylene glycol, polyvinyl alcohol, polyvinyl pyrrolidone and petroleum. Suitable forms of the composition include a solid, a paste, an ointment, a gel, a liquid, an aerosol, a mist, a polymer, a film, an emulsion, and a suspension. In some cases, the composition is incorporated into or coated onto a contact lens. Optionally, the composition is present at a concentration of 0.1-10% (mg/ml).

All polynucleotides and polypeptides of the invention are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

An “isolated nucleic acid” is a nucleic acid, the structure of which is not identical to that of any naturally occurring nucleic acid, or to that of any fragment of a naturally occurring genomic nucleic acid spanning more than three separate genes. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybridgene, i.e., a gene encoding a fusion protein. Isolated nucleic acid molecules according to the present invention further include molecules produced synthetically, as well as any nucleic acids that have been altered chemically and/or that have modified backbones. Isolated nucleic acid molecules also include messenger ribonucleic acid (mRNA) molecules.

Although the phrase “nucleic acid molecule” primarily refers to the physical nucleic acid molecule and the phrase “nucleic acid sequence” refers to the sequence of the nucleotides the nucleic acid molecule, the two phrases can be used interchangeably.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent inflammation (e.g., ocular inflammation) in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a series of flow cytometry graphs and a bar chart showing that a significantly increased number of cells expressed forkhead box P3 (Foxp3) and CD25 when CD4+CD25− T cells were activated in the presence of TSP-1 peptide (4N1K) as compared to the control peptide (4NGG). The cells also increased synthesis of immunosuppressive cytokine transforming growth factor beta-1 (TGFβ1).

FIG. 2A is a flow cytometry graph showing that TSP-peptide (4N1K)-treated mice contained increased numbers of Foxp3 expressing T_{reg} cells, as compared to the control peptide. FIG. 2B is a flow cytometry graph showing that TSP-peptide (4N1K)-treated mice contained reduced numbers of interleukin 17 (IL-17), as compared to the control peptide. FIG. 2C is a flow cytometry graph showing that TSP-peptide (4N1K)-treated mice contained reduced numbers of interferon gamma (IFN-γ)-expressing CD4 effector cells, as compared to the control peptide.

FIG. 3 is a line graph and a series of photomicrographs demonstrating that intraperitoneal TSP-peptide (4N1K) treatment resulted in significantly reduced disease severity, as compared to the control peptide. Representative fundus images are shown as seen on day 26 post-immunization.

FIG. 4 is a line graph and a series of photomicrographs demonstrating that topical TSP-1 peptide (4N1K) treatment resulted in significantly reduced disease severity, as compared to the control peptide.

FIG. 5 is a bar chart showing the changes in the message for transcriptional factors associated with pro-inflammatory Tₕ17 cells and anti-inflammatory T_{reg} cells in the lymph nodes of TSP-1 peptide treated mice relative to those treated with the control peptide.

FIG. 6 is a schematic representation of the structure of thrombospondin-1.

### DETAILED DESCRIPTION

Chronic inflammatory conditions are associated with many autoimmune diseases as well as transplantation rejection. Conventional therapies to treat chronic inflammation include non-specific immune-suppression, which is typically achieved via steroids or cytotoxic agents. Specifically, inflammation of the ocular and adnexal tissues can occur by a variety of mechanisms and is associated, either primarily or secondarily, with a large number of disease conditions. Current treatments for inflammation of these tissues involve the systemic administration of antibiotics, steroids, and immune-system inhibitors. The difficulty of using these systemic drugs becomes apparent through damaging long-term side effects in the case of steroids, long-term drug resistance in the case of antibiotics, or insufficient long-term persistence at the target site in the case of signaling inhibitors. Moreover, the systemic inhibition of signaling within the immune system can have deleterious outcomes for individuals already afflicted with disease, whose susceptibility to additional complications is increased as a result of the systemic use of these treatments.

The present invention overcomes these obstacles by the administration of an anti-inflammatory peptide derived from thrombospondin-1 (TSP-1). As described herein, the anti-inflammatory TSP-1 peptide increases the population of anti-inflammatory regulatory T cells (T_{reg}) and decreases the population of pro-inflammatory T helper 17 (Tₕ17) cells in an ocular or adnexal tissue in a subject. Thus, the compositions and methods described herein are useful in the treatment of any disease associated with Tₕ1 and Tₕ17 inflammatory T cells.

T helper 17 cells (Tₕ17) are a subset of T helper cells that produce interleukin 17 (IL-17). IL-17 is a primarily T-cell-derived pro-inflammatory cytokine with an increased expression detected in chronic inflammatory conditions such as rheumatoid arthritis (Kotake S, et al. 1999. J Clin Invest, 103: 1345-52; Chabaud M, et al. 1999. Arthritis Rheum, 42: 963-70), allograft rejection (Antonysamy M A, et al. 1999. J Immunol, 162: 577-84; Hsieh H G, et al. 2001. Transpl Int, 14: 287-98), multiple sclerosis (Kurasawa K, et al. 2000. Arthritis Rheum, 43: 2455-63), psoriasis (Chiricozzi A, et al. 2011. J Invest Dermatol, 131: 677-87), ankylosing spondylitis (Mei Y, et al. 2011. Clin Rheumatol, 30: 269-73), uveitis (Hamzaoui K, et al. 2002. Scand J Rheumatol, 31: 205-10; Chi W, et al. 2010. Mol V is, 16: 880-6), and Sjögren's syndrome (Katsifis G E, et al. 2009. Am J Pathol, 175: 1167-77). Excessive numbers of Tₕ17 cells play a key role in inflammation and autoimmune disease (e.g., multiple sclerosis). For example, Tₕ17 cells are involved in psoriasis, autoimmune uveitis, juvenile diabetes, rheumatoid arthritis, and Crohn's disease.

Regulatory T cells (T_{reg} or suppressor T cells), are a subpopulation of T cells which downregulate the immune system, maintain tolerance to self-antigens, and help prevent autoimmunity. The subset of lymphocytes known as regulatory T cells includes T cells identified by the markers CD4+CD25+Foxp3+. These cells are important in maintaining the homeostatic balance of the immune system. Deficiencies in this subset are linked to unfavorable immune responses such as autoimmune diseases, allergy and allograft rejection. Under these conditions, an immune response to certain antigens leads to the generation of antigen-specific effector memory cells that can overwhelm the available regulation provided by T_{reg} cells.

During an immune response, the differentiation of activated T cells to express transcription factor RORγt leads to the production of pro-inflammatory IL-17-producing Tₕ17 effector cells, while the expression of forkhead box P3 (Foxp3) leads to the production of anti-inflammatory transforming growth factor beta (TGF-β)-producing T_{reg} cells. A predominance of the Tₕ17 subset over the T_{reg} subset results in a pro-inflammatory outcome.

The methods described herein inhibit the population of inflammatory interleukin-17 (IL-17)-producing T cells or neutralize the IL-17 produced by these cells, thereby reducing chronic inflammatory conditions. The methods of the invention also expand the population of T_{reg} cells. By reducing the population of pro-inflammatory effector cells and increasing the population of anti-inflammatory regulatory T cells, the methods of the invention downregulate aberrant and detrimental immune responses.

The anti-inflammatory peptide described herein is derived from a large glycoprotein, thrombospondin-1 (TSP-1). TSP-1 binds multiple receptors and exerts different biological effects depending on the cell type. This molecule is critical to the regulation of ocular inflammatory responses, as it affects ocular antigen presenting cells (APCs) (Saban D R, et al. 2010. J Immunol, 185: 4691-7; Ng T F, et al. 2009. Invest Ophthalmol V is Sci, 50: 5472-8; Masli S, et al. 2006. Int Immunol, 18: 689-99; Zamiri P, et al. 2005. Invest Ophthalmol V is Sci, 46: 908-19; Masli S, et al. 2002. J Immunol, 168: 2264-73). Moreover, increased expression of TSP-1 by ocular antigen presenting cells facilitates the induction of Foxp3-expressing and TGF-β-producing T_{reg} cells (Mash S. T B. 2010. In The Association of Research in Vision and Opthalmology (ARVO). Ft. Lauderdale, Fla.; Mir F T B, Masli S. 2011. In The Association of Research in Vision and Opthalmology (ARVO). Ft. Lauderdale, Fla.). This T_{reg} subset is induced via the engagement of CD47 receptors on activated T cells, as demonstrated by in vitro experiments. Similarly, the T_{reg} subset is induced in vitro via Foxp3 in human T cells upon ligation of CD47 (Grimbert P, et al. 2006. J Immunol, 177: 3534-41).

The inflammatory disease prevented or treated by the methods described herein may be any acute or chronic inflammatory disease. In one aspect, the inflammatory disease is a non-ocular inflammatory disease selected from the group consisting of autoimmune diseases such as psoriasis, rheumatoid arthritis, inflammatory bowel disease, multiple sclerosis, Sjögren's syndrome, vasculitis, ankylosing spondylitis and transplant rejection disease (i.e., allograft rejection). The inflammatory disease is not cancer or a tumor. Accordingly, the method does not involve intravascular administration into vessels infiltrating a tumor or tumor metastasis. Preferably, the inflammatory disease is an ocular inflammatory disease selected from the group consisting of dry eye disease, uveitis, conjunctivitis, and keratitis.

As described in Examples 2 and 3 below, the administration of the TSP-peptide described herein has an anti-inflammatory effect regardless of the antigen used in experimental models. Therefore, the invention is applicable to a broader range of inflammatory diseases beyond those affecting the eye.

Prior to the invention described herein, most anti-inflammatory strategies included the use of immunosuppressive pharmacologic agents such as glucocorticoids, cyclosporine A, cyclophosphamide, methotrexate or biologic agents such as antibodies that neutralize inflammatory cytokines such as tumor necrosis factor alpha (TNF-α), IL-1β, and IL-17. Prior to the invention described herein, other anti-inflammatory strategies included peptide-based therapeutic vaccines for antigen-specific immune modulation or induction of immunologic tolerance.

Most currently available anti-inflammatory therapeutic agents are known to result in non-specific immune-suppression, which is associated with complications in the treatment of chronic inflammation. These adverse side effects range from infections and tumor development to the disruption of natural regulatory mechanisms. While the peptide-based therapeutic vaccines modulate the immune response in an antigen-specific manner, the strategies require the appropriate selection of an epitope from an antigen and its corresponding peptide. Polymorphism of genes encoding human major histocompatibility complex (MHC) molecules presents a challenge for peptide vaccine design. Moreover, effective peptides longer than 20 amino acids are associated with adverse events due to their ability to cross-link receptors on innate cells.

By contrast, the TSP-1 peptide described herein overcomes the challenges associated with vaccine design and is 10 amino acids in length, thereby minimizing the possibility of adverse side effects. Also, TSP-1 is a well-conserved molecule across species. Therefore, it is not likely that TSP-1 will elicit an immune reaction against itself upon repeated administration.

### Thrombospondin-1

Human Thrombospondin-1 (THBS1 or TSP-1) has the following mRNA sequence
(SEQ ID NO: 2; GenBank Accession No. NM_003246 (GI:40317625),
incorporated herein by reference).

| | |
|---|---|
| 1 | agccgctgcg cccgagctgg cctgcgagtt cagggctcct gtcgctctcc aggagcaacc |
| 61 | tctactccgg acgcacaggc attccccgcg cccctccagc cctcgccgcc ctcgccaccg |
| 121 | ctcccggccg ccgcgctccg gtacacacag gatccctgct gggcaccaac agctccacca |
| 181 | tggggctggc ctggggacta ggcgtcctgt tcctgatgca tgtgtgtggc accaaccgca |
| 241 | ttccagagtc tggcggagac aacagcgtgt ttgacatctt tgaactcacc ggggccgccc |
| 301 | gcaaggggtc tgggcgccga ctggtgaagg gccccgaccc ttccagccca gctttccgca |
| 361 | tcgaggatgc caacctgatc ccccctgtgc ctgatgacaa gttccaagac ctggtggatg |
| 421 | ctgtgcgggc agaaaagggt ttcctccttc tggcatccct gaggcagatg aagaagaccc |
| 481 | ggggcacgct gctggccctg gagcggaaag accactctgg ccaggtcttc agcgtggtgt |
| 541 | ccaatggcaa ggcgggcacc ctggacctca gcctgaccgt ccaaggaaag cagcacgtgg |
| 601 | tgtctgtgga agaagctctc ctggcaaccg gccagtggaa gagcatcacc ctgtttgtgc |
| 661 | aggaagacag ggcccagctg tacatcgact gtgaaaagat ggagaatgct gagttggacg |
| 721 | tccccatcca aagcgtcttc accagagacc tggccagcat cgccagactc cgcatcgcaa |
| 781 | aggggggcgt caatgacaat ttccaggggg tgctgcagaa tgtgaggttt gtctttggaa |
| 841 | ccacaccaga agacatcctc aggaacaaag gctgctccag ctctaccagt gtcctcctca |
| 901 | cccttgacaa caacgtggtg aatggttcca gccctgccat ccgcactaac tacattggcc |
| 961 | acaagacaaa ggacttgcaa gccatctgcg gcatctcctg tgatgagctg tccagcatgg |
| 1021 | tcctggaact caggggcctg cgcaccattg tgaccacgct gcaggacagc atccgcaaag |
| 1081 | tgactgaaga gaacaaagag ttggccaatg agctgaggcg gcctccccta tgctatcaca |
| 1141 | acggagttca gtacagaaat aacgaggaat ggactgttga tagctgcact gagtgtcact |
| 1201 | gtcagaactc agttaccatc tgcaaaaagg tgtcctgccc catcatgccc tgctccaatg |
| 1261 | ccacagttcc tgatggagaa tgctgtcctc gctgttggcc cagcgactct gcggacgatg |
| 1321 | gctggtctcc atggtccgag tggacctcct gttctacgag ctgtggcaat ggaattcagc |
| 1381 | agcgcggccg ctcctgcgat agcctcaaca accgatgtga gggctcctcg gtccagacac |
| 1441 | ggacctgcca cattcaggag tgtgacaaga gatttaaaca ggatggtggc tggagccact |
| 1501 | ggtccccgtg gtcatcttgt tctgtgacat gtggtgatgg tgtgatcaca aggatccggc |
| 1561 | tctgcaactc tcccagcccc cagatgaacg ggaaaccctg tgaaggcgaa gcgcgggaga |
| 1621 | ccaaagcctg caagaaagac gcctgcccca tcaatggagg ctggggtcct tggtcaccat |
| 1681 | gggacatctg ttctgtcacc tgtggaggag gggtacagaa acgtagtcgt ctctgcaaca |
| 1741 | accccacacc ccagtttgga ggcaaggact gcgttggtga tgtaacagaa aaccagatct |
| 1801 | gcaacaagca ggactgtcca attgatggat gcctgtccaa tccctgcttt gccggcgtga |
| 1861 | agtgtactag ctaccctgat ggcagctgga aatgtggtgc ttgtccccct ggttacagtg |
| 1921 | gaaatggcat ccagtgcaca gatgttgatg agtgcaaaga agtgcctgat gcctgcttca |
| 1981 | accacaatgg agagcaccgg tgtgagaaca cggaccccgg ctacaactgc ctgccctgcc |
| 2041 | ccccacgctt caccggctca cagcccttcg gccagggtgt cgaacatgcc acggccaaca |
| 2101 | aacaggtgtg caagccccgt aacccctgca cggatgggac ccacgactgc aacaagaacg |
| 2161 | ccaagtgcaa ctacctgggc cactatagcg accccatgta ccgctgcgag tgcaagcctg |
| 2221 | gctacgctgg caatggcatc atctgcgggg aggacacaga cctggatggc tggcccaatg |
| 2281 | agaacctggt gtgcgtggcc aatgcgactt accactgcaa aaaggataat tgccccaacc |
| 2341 | ttcccaactc agggcaggaa gactatgaca aggatggaat tggtgatgcc tgtgatgatg |
| 2401 | acgatgacaa tgataaaatt ccagatgaca gggacaactg tccattccat tacaacccag |
| 2461 | ctcagtatga ctatgacaga gatgatgtgg gagaccgctg tgacaactgt ccctacaacc |
| 2521 | acaacccaga tcaggcagac acagacaaca atggggaagg agacgcctgt gctgcagaca |
| 2581 | ttgatggaga cggtatcctc aatgaacggg acaactgcca gtacgtctac aatgtggacc |
| 2641 | agagagacac tgatatggat ggggttggag atcagtgtga caattgcccc ttggaacaca |
| 2701 | atccggatca gctggactct gactcagacc gcattggaga tacctgtgac aacaatcagg |
| 2761 | atattgatga agatggccac cagaacaatc tggacaactg tccctatgtg cccaatgcca |
| 2821 | accaggctga ccatgacaaa gatggcaagg gagatgcctg tgaccacgat gatgacaacg |
| 2881 | atggcattcc tgatgacaag gacaactgca gactcgtgcc caatcccgac cagaaggact |
| 2941 | ctgacggcga tggtcgaggt gatgcctgca aagatgattt tgaccatgac agtgtgccag |
| 3001 | acatcgatga catctgtcct gagaatgttg acatcagtga gaccgatttc cgccgattcc |
| 3061 | agatgattcc tctggacccc aaagggacat cccaaaatga ccctaactgg gttgtacgcc |
| 3121 | atcagggtaa agaactcgtc cagactgtca actgtgatcc tggactcgct gtaggttatg |
| 3181 | atgagtttaa tgctgtggac ttcagtggca ccttcttcat caacaccgaa agggacgatg |
| 3241 | actatgctgg atttgtcttt ggctaccagt ccagcagccg cttttatgtt gtgatgtgga |
| 3301 | agcaagtcac ccagtcctac tgggacacca accccacgag ggctcaggga tactcgggcc |
| 3361 | tttctgtgaa agttgtaaac tccaccacag ggcctggcga gcacctgcgg aacgccctgt |
| 3421 | ggcacacagg aaacacccct ggccaggtgc gcaccctgtg gcatgaccct cgtcacatag |
| 3481 | gctggaaaga tttcaccgcc tacagatggc gtctcagcca caggccaaag acgggtttca |
| 3541 | ttagagtggt gatgtatgaa gggaagaaaa tcatggctga ctcaggaccc atctatgata |
| 3601 | aaacctatgc tggtggtaga ctagggttgt ttgtcttctc tcaagaaatg gtgttcttct |
| 3661 | ctgacctgaa atacgaatgt agagatccct aatcatcaaa ttgttgattg aaagactgat |
| 3721 | cataaaccaa tgctggtatt gcaccttctg gaactatggg cttgagaaaa cccccaggat |
| 3781 | cacttctcct tggcttcctt cttttctgtg cttgcatcag tgtggactcc tagaacgtgc |
| 3841 | gacctgcctc aagaaaatgc agttttcaaa aacagactca gcattcagcc tccaatgaat |
| 3901 | aagacatctt ccaagcatat aaacaattgc tttggtttcc ttttgaaaaa gcatctactt |
| 3961 | gcttcagttg ggaaggtgcc cattccactc tgcctttgtc acagagcagg gtgctattgt |
| 4021 | gaggccatct ctgagcagtg gactcaaaag cattttcagg catgtcagag aagggaggac |
| 4081 | tcactagaat tagcaaacaa aaccaccctg acatcctcct tcaggaacac ggggagcaga |
| 4141 | ggccaaagca ctaaggggag ggcgcatacc cgagacgatt gtatgaagaa aatatggagg |
| 4201 | aactgttaca tgttcggtac taagtcattt tcaggggatt gaaagactat tgctggattt |
| 4261 | catgatgctg actggcgtta gctgattaac ccatgtaaat aggcacttaa atagaagcag |
| 4321 | gaaagggaga caaagactgg cttctggact tcctccctga tccccaccct tactcatcac |
| 4381 | ctgcagtggc cagaattagg gaatcagaat caaaccagtg taaggcagtg ctggctgcca |
| 4441 | ttgcctggtc acattgaaat tggtggcttc attctagatg tagcttgtgc agatgtagca |
| 4501 | ggaaaatagg aaaacctacc atctcagtga gcaccagctg cctcccaaag gaggggcagc |
| 4561 | cgtgcttata tttttatggt tacaatggca caaaattatt atcaacctaa ctaaaacatt |
| 4621 | ccttttctct tttttcctga attatcatgg agttttctaa ttctctcttt tggaatgtag |
| 4681 | atttttttta aatgctttac gatgtaaaat atttattttt tacttattct ggaagatctg |
| 4741 | gctgaaggat tattcatgga acaggaagaa gcgtaaagac tatccatgtc atctttgttg |
| 4801 | agagtcttcg tgactgtaag attgtaaata cagattattt attaactctg ttctgcctgg |
| 4861 | aaatttaggc ttcatacgga aagtgtttga gagcaagtag ttgacattta tcagcaaatc |
| 4921 | tcttgcaaga acagcacaag gaaaatcagt ctaataagct gctctgcccc ttgtgctcag |
| 4981 | agtggatgtt atgggattct ttttttctct gttttatctt ttcaagtgga attagttggt |
| 5041 | tatccatttg caaatgtttt aaattgcaaa gaaagccatg aggtcttcaa tactgtttta |
| 5101 | ccccatccct tgtgcatatt tccagggaga aggaaagcat atacactttt ttctttcatt |
| 5161 | tttccaaaag agaaaaaaat gacaaaaggt gaaacttaca tacaaatatt acctcatttg |
| 5221 | ttgtgtgact gagtaaagaa tttttggatc aagcggaaag agtttaagtg tctaacaaac |
| 5281 | ttaaagctac tgtagtacct aaaaagtcag tgttgtacat agcataaaaa ctctgcagag |
| 5341 | aagtattccc aataaggaaa tagcattgaa atgttaaata caatttctga aagttatgtt |
| 5401 | ttttttctat catctggtat accattgctt tatttttata aattattttc tcattgccat |
| 5461 | tggaatagat atctcagatt gtgtagatat gctatttaaa taatttatca ggaaatactg |
| 5521 | cctgtagagt tagtatttct atttttatat aatgtttgca cactgaattg aagaattgtt |
| 5581 | ggttttttct tttttttgtt ttgttttttt tttttttttt ttttgctttt gacctcccat |
| 5641 | ttttactatt tgccaatacc tttttctagg aatgtgcttt tttttgtaca catttttatc |
| 5701 | cattttacat tctaaagcag tgtaagttgt atattactgt ttcttatgta caaggaacaa |
| 5761 | caataaatca tatggaaatt tatatttata aaaaaaaaaa aaaaaaaaaa aaaaaaaaaa |

Human Thrombospondin-1 (THBS1 or TSP-1) has the following protein
sequence (SEQ ID NO: 3; GenBank Accession No. AAI36471 (GI:223460844),
incorporated herein by reference).

| | |
|---|---|
| 1 | mglawglgvl flmhvcgtnr ipesggdnsv fdifeltgaa rkgsgrrlvk gpdpsspafr |
| 61 | iedanlippv pddkfqdlvd avraekgfll laslrqmkkt rgtllalerk dhsgqvfsvv |
| 121 | sngkagtldl sltvqgkqhv vsveeallat gqwksitlfv qedraglyid cekmenaeld |
| 181 | vpiqsvftrd lasiarlria kggvndnfqg vlqnvrfvfg ttpedilrnk gcssstsvll |
| 241 | tldnnvvngs spairtnyig hktkdlqaic giscdelssm vlelrglrti vttlqdsirk |
| 301 | vteenkelan elrrpplcyh ngvqyrnnee wtvdsctech cqnsvtickk vscpimpcsn |
| 361 | atvpdgeccp rcwpsdsadd gwspwsewts cstscgngiq qrgrscdsln nrcegssvqt |
| 421 | rtchiqecdk rfkqdggwsh wspwsscsvt cgdgvitrir lcnspspqmn gkpcegeare |
| 481 | tkackkdacp inggwgpwsp wdicsvtcgg gvqkrsrlcn nptpqfggkd cvgdvtenqi |
| 541 | cnkqdcpidg clsnpcfagv kctsypdgsw kcgacppgys gngiqctdvd eckevpdacf |
| 601 | nhngehrcen tdpgynclpc pprftgsqpf gqgvehatan kqvckprnpc tdgthdcnkn |
| 661 | akcnylghys dpmyrceckp gyagngiicg edtdldgwpn enlvcvanat yhckkdncpn |
| 721 | lpnsgqedyd kdgigdacdd dddndkipdd rdncpfhynp aqydydrddv gdrcdncpyn |
| 781 | hnpdqadtdn ngegdacaad idgdgilner dncqyvynvd qrdtdmdgvg dqcdncpleh |
| 841 | npdqldsdsd rigdtcdnnq didedghqnn ldncpyvpna nqadhdkdgk gdacdhdddn |
| 901 | dgipddkdnc rlvpnpdqkd sdgdgrgdac kddfdhdsvp diddicpenv disetdfrrf |
| 961 | qmipldpkgt sqndpnwvvr hqgkelvqtv ncdpglavgy defnavdfsg tffinterdd |
| 1021 | dyagfvfgyq sssrfyvvmw kqvtqsywdt nptraqgysg lsvkvvnstt gpgehlrnal |
| 1081 | whtgntpgqv rtlwhdprhi gwkdftayrw rlshrpktgf irvvmyegkk imadsgpiyd |
| 1141 | ktyaggrlgl fvfsqemvff sdlkyecrdp |

The C-terminal peptide of human thrombospondin-1 described herein (4N1K) has the following sequence: KRFYVVMWKK (SEQ ID NO: 1). Other useful CD47-binding peptides include FIRVVMYEGKK (7N3, residues 1102-1112 of mature TSP1: SEQ ID NO:4), RFYVVMWK (4N1-1, 1016-1024; SEQ ID NO:5), and KRFYVVMWKK (4N1K, 4N1 flanked with 2 Lys residues; SEQ ID NO:6) (Barazi et al., 2002, J. Biol. Chem. Vol. 277:42859-42866; hereby incorporated by reference).

A schematic representation of the structure of thombospondin-1 (TSP-1) is provided in FIG. 6. Thrombospondin 1 (TSP-1 or THBS1) is a protein that is encoded by the thbs1 gene in humans. Thrombospondin 1 is a subunit of a disulfide-linked homotrimeric protein. This protein is an adhesive glycoprotein that mediates cell-to-cell and cell-to-matrix interactions. As shown in FIG. 6, TSP-1 has several binding sites, including a binding site for latent TGF-13. The location of this binding site is within the central type 1 repeats. By contrast, the TSP-1 derived peptide described herein is located within the C-terminal domain. Thrombospondin 1 binds to fibrinogen, fibronectin, laminin, type V collagen, and integrins alpha-V/beta-1.

### Ocular and Adnexal Tissues:

Ocular tissues or compartments that contact the compositions comprised by the present invention include, but are not limited to, the cornea, aqueous humor, iris, and sclera. The term “adnexal” is defined in general terms as the appendages of an organ. In the present invention, adnexal defines a number of tissues or surfaces that are in immediate contact with the ocular surface but are not, by definition, comprised by the ocular surface. Exemplary adnexal tissues include, but are not limited to, the eyelids, lacrimal glands, and extraocular muscles. Topical administration of the presently invented compositions contact the following tissues and structures within the eyelid: skin, subcutaneous tissue, orbicularis oculi, orbital septum, tarsal plates, palpebral conjuntiva, and meibomian glands. The adnexal tissues comprise all subdivisions of the lacrimal glands, including the orbital and palpebral portions, as well as all tissues contacted by these glands. Extraocular muscles belonging to this category of adnexal tissues include, but are not limited to, the superior and inferior rectus, lateral and medial rectus, and superior and inferior oblique muscles. Compositions comprised by the present invention are applied topically and contact these tissues either alone, or in combination with ocular tissues. Topical application of drugs may enter blood circulation via blood vessels in the conjunctiva. Therefore although exposure of other organs to smaller amounts of a drug applied in this manner cannot be ruled out, the amount is typically well below an amount that would lead to systemic side effects.

### Pharmaceutically-Appropriate Carriers

The active agent(s) may be combined with a pharmaceutically-acceptable carrier (e.g., antioxidants, wetting agents, buffers, and tonicity adjusters). Exemplary compounds incorporated to facilitate and expedite local delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alpha-terpeneol, 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist.

Optionally, the composition further contains a compound selected from the group consisting of a physiological acceptable salt, poloxamer analogs with carbopol, carbopol/hydroxypropyl methyl cellulose (HPMC), carbopol-methyl cellulose, N-acetyl cysteine, carboxymethylcellulose (CMC), hyaluronic acid, cyclodextrin, and petroleum.

Examples of suitable pharmaceutical excipients include, water, glucose, sucrose, lactose, glycol, ethanol, glycerol monostearate, gelatin, rice, starch, flour, chalk, sodium stearate, malt, sodium chloride and the like. The pharmaceutical compositions of the present invention can take the form of solutions, capsules, tablets, creams, gels, powders, sustained release formulations and the like. The composition can be formulated as a suppository, with traditional binders and carriers such as triglycerides (see Remington: The Science and Practice of Pharmacy by Alfonso R. Gennaro, 2003, 21.sup.th edition, Mack Publishing Company). Such compositions contain a therapeutically effective amount of the therapeutic composition, together with a suitable amount of carrier so as to provide the form for proper administration to the subject. The formulations are designed so as to suit the mode of administration and the target site of action (e.g., a particular organ or cell type).

Various delivery systems are known and can be used to the active agent(s). The pharmaceutical composition of the present invention can be administered by any suitable route including, intravenous or intramuscular injection, intraventricular or intrathecal injection (for central nervous system administration), orally, topically, subcutaneously, subconjunctivally, or via intranasal, intradermal, sublingual, vaginal, rectal or epidural routes.

Preferably, the pharmaceutical compositions according to the present invention are formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

### Human Dose Range

Human equivalent does (HED) and dose ranges are calculated using methods known in the art, e.g, Reagan-Shaw et al., 2007, FASEB J. 22659-661; hereby incorporated by reference). For example, 10 micrograms of peptide were used per mouse (typical weight 20-30 gm). Therefore, using the formula described in the reference provided above, an HED of 1.62-2.4 mg per 60 kg human is calculated.

### Preferred Excipients/Formulations

In the experiments described herein, peptide preparations were water based, as peptides are soluble in water. Both topical and i.p. applications were equally effective. These observations indicate that topically applied peptide through nasolacrimal drainage effectively are as good as systemic administration.

In a preferred example, the peptide is delivered in the form of a conventional eye drop. The target population of CD47-binding TSP peptides is immune effector cells such as inflammatory T cells and antigen presenting cells (either infiltrating the tissue or circulating), which eliminates the need for retaining it locally in the eye for extended periods of time (unlike some other drugs where cellular targets can be within the ocular tissue only). If local retention for extended periods of time is desired, topical peptide delivery in humans is also accomplished via hydrogel application with a contact lens.

The formulation preferably comprises a pharmaceutically-acceptable carrier that assists in retention of the formulation on the ocular surface. Excipients with either increased viscosity or bioadhesive properties are commonly used to increase this dwell time. Exemplary excipients include carbopol gels, cellulose derivatives, detran, gelatin glycerin, polyethylene glycol, poloxamer 407, polysorbate 80, propylene glycol, polyvinyl alcohol and polyvinyl pyrrolidone. Other suitable carriers that assist in retention of the formulation on the ocular surface are described in Kuno and Fujii, 2011 Polymers, 3: 193-221; incorporated herein by reference.

For example, carboxymethylcellulose (CMC), a high-molecular-weight polysaccharide, is one of the most common viscous polymers used in artificial tears to achieve their prolonged residence time on the ocular surface (See, e.g., Garrett, et al., 2007 IOVS, 48(4): 1559-1567; incorporated herein by reference). The physical properties of CMC, such as its viscous and mucoadhesive properties, contribute to its prolonged retention time on the ocular surface.

### Drug Delivery by Contact Lens

The invention comprises a contact lens and a composition that inhibits an activity of an inflammatory interleukin-1 cytokine. For example, the composition is incorporated into or coated onto said lens. The composition is chemically bound or physically entrapped by the contact lens polymer. Alternatively, a color additive is chemically bound or physically entrapped by the polymer composition that is released at the same rate as the therapeutic drug composition, such that changes in the intensity of the color additive indicate changes in the amount or dose of therapeutic drug composition remaining bound or entrapped within the polymer. Alternatively, or in addition, an ultraviolet (UV) absorber is chemically bound or physically entrapped within the contact lens polymer. The contact lens is either hydrophobic or hydrophilic. Such an approach may lead to enhanced efficacy of peptides if retained locally as compared to potential systemic absorption via blood circulation.

### EXAMPLES

### Example 1

### C-Terminal Peptide of TSP-1 Binds CD47 Receptor and Expands T_{reg} Cells In Vitro

The results presented herein demonstrate that a small peptide (10 amino acids) from the C-terminal portion of TSP-1 binds CD47 receptor (4N1K) and effectively induces T_{reg} cells in vitro. The C-Terminal peptide of TSP-1 utilized in the experiments described herein has the following sequence: KRFYVVMWKK (SEQ ID NO: 1).

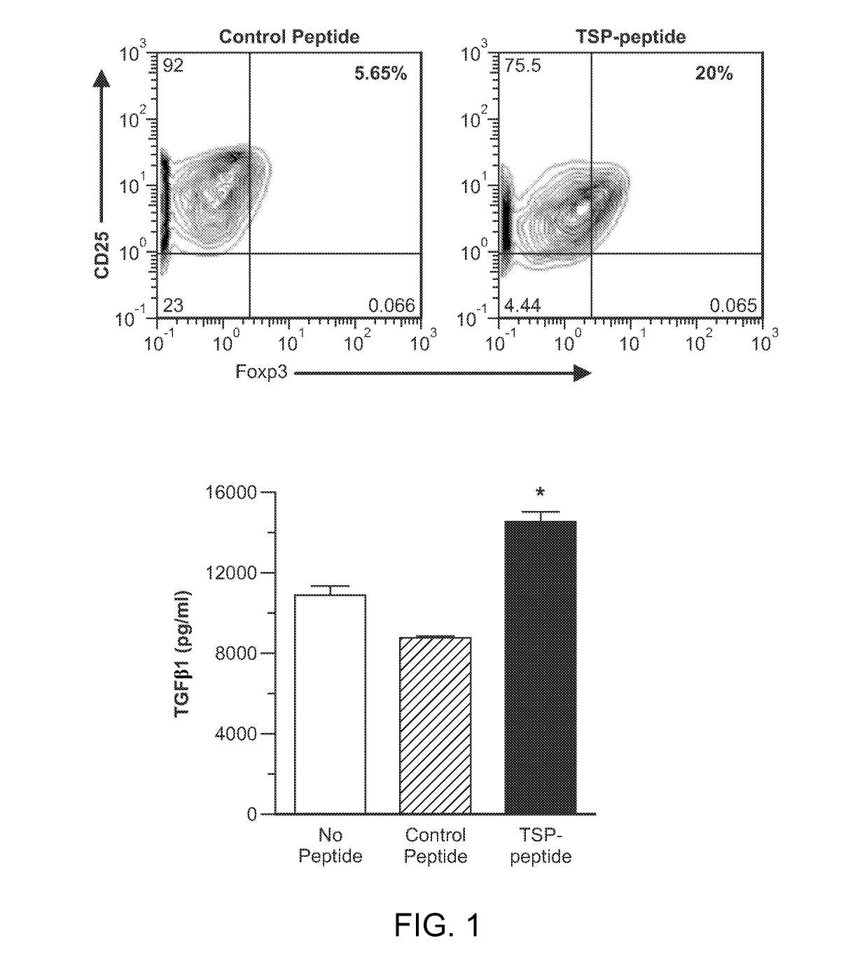

As shown in FIG. 1, a significantly increased number of cells expressed Foxp3 and CD25 when CD4+CD25− T cells were activated in the presence of TSP-peptide (4N1K) as compared to the control peptide (4NGG) (Masli S. T B. 2010. In The Association of Research in Vision and Opthalmology (ARVO). Ft. Lauderdale, Fla.). The cells also increased the synthesis of immunosuppressive cytokine TGF-β1.

Purified CD4+CD25− T cells were stimulated with plate-bound anti-CD3 antibody for 48 hr at 37 degrees C. in the presence of 10 microM TSP-peptide or control peptide. Activated T cells were washed and subjected to 2 cycles of amplification, 48 hr each, in the presence of T cell growth factor IL-2 (50 U/ml). Proliferated T cells were then stained using fluorochrome-conjugated antibodies to label surface CD4 and CD25 and intracellular Foxp3. Stained cells were analyzed using a flow cytometer. Culture supernatants collected from cells after initial 48 hr culture were tested for the content of TGFbeta using an ELISA.

### Example 2

### C-Terminal Peptide of TSP-1 Expands T_{reg} Cells and Inhibits IL-17 and IFN-γ Inflammatory Effector Cells In Vivo

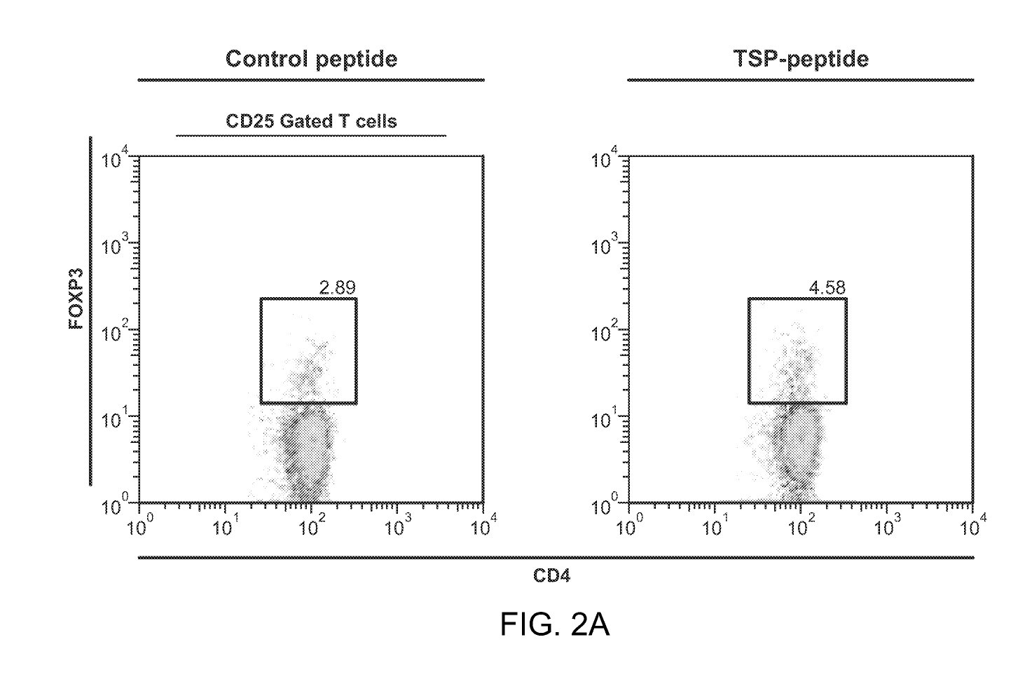

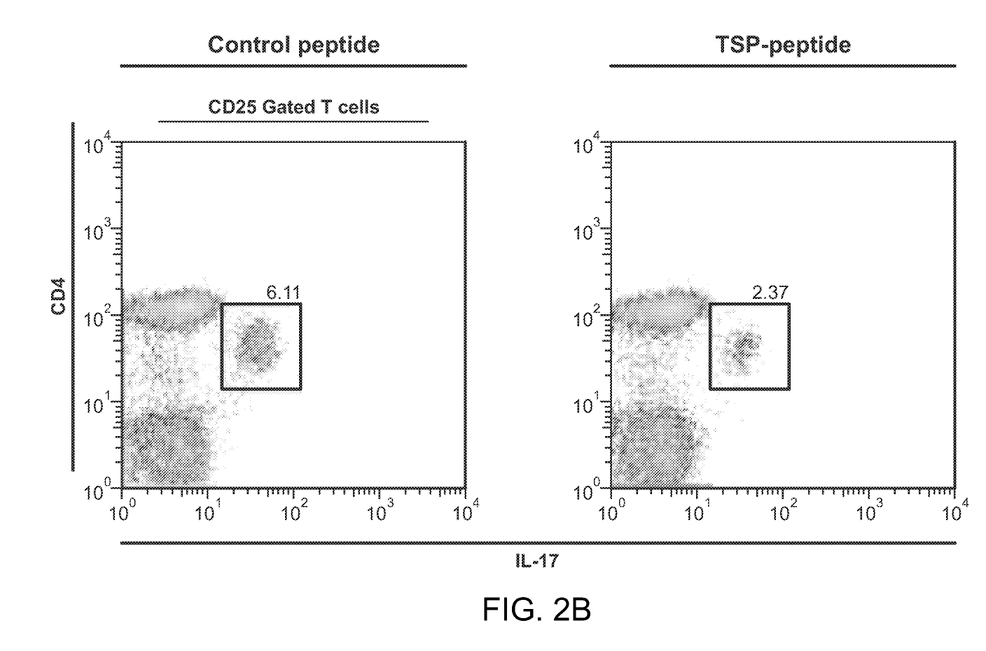

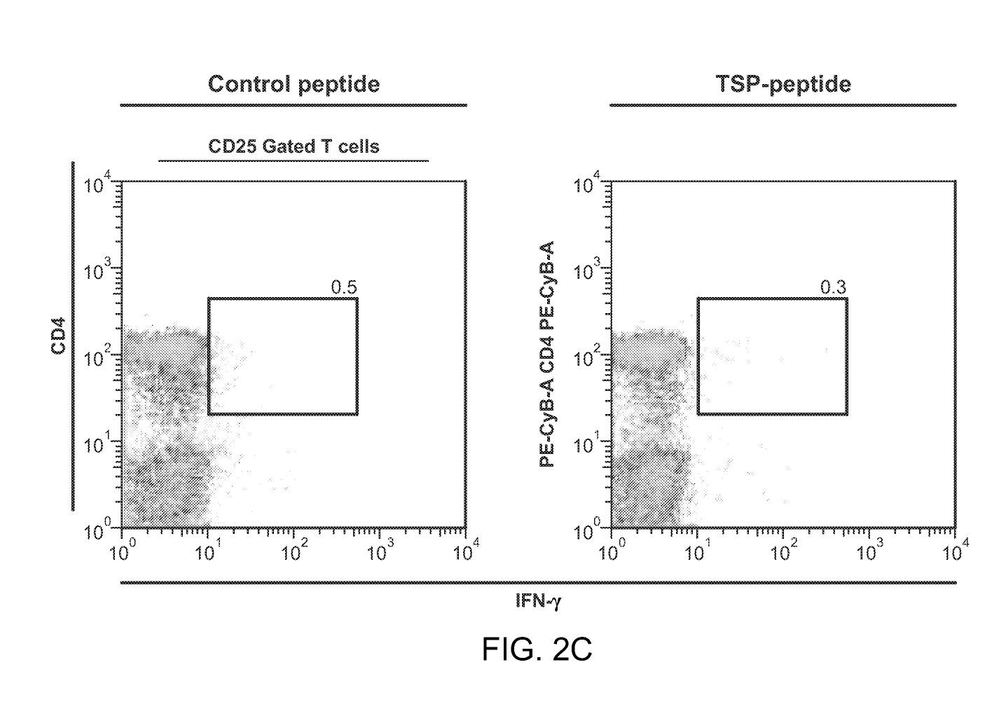

To determine if such an expansion of T_{reg} cells by TSP-peptide is possible in vivo, and whether it can reduce the typical IL-17 or IFNγ-producing inflammatory effectors, mice were immunized with an ovalbumin (ova) antigen in Complete Freund's Adjuvant (CFA). This is a commonly used experimental approach to generate antigen-specific inflammatory effectors. These ova-immunized mice were treated with either control or TSP-peptide (10 μg; SEQ ID NO:1) on day 1, 3 and 5 post-immunization. On day 7 post-immunization, the CD4+ T cells derived from the spleen and lymph node of these mice were analyzed by flow cytometry to determine the numbers of Foxp3+ T_{reg} cells and the expression patterns of the inflammatory cytokines, IL-17 and IFN-λ. As shown in FIG. 2A-C, mice treated with the TSP-1 peptide contained increased numbers of Foxp3-expressing T_{reg} cells (FIG. 2A) and reduced numbers of IL-17 (FIG. 2B) and IFN-γ-expressing CD4 effectors (FIG. 2C), as compared to mice treated with the control peptide.

Lymph nodes were harvested from immunized mice treated with either control or TSP-peptide 7 days post-immunization. Lymph node cells were cultured in the presence of PMA (10 ng/ml), ionomycin (200 ng/ml) and Brefeldin A for 20 hr at 37 degrees C. Cell surface CD4 and CD25 and intracellular Foxp3, IFNgamma and IL-17 were detected using fluorochrome-conjugated antibodies followed by flow cytometric analysis.

### Example 3

### C-Terminal Peptide of TSP-1 Reduces Disease Severity in an Experimental Mouse Model of Autoimmune Uveitis

The expansion of T_{reg} cells and the reduction of inflammatory effector cells was evaluated in an experimental mouse model of autoimmune uveitis to determine if the TSP-1 peptide reduced disease severity. In this model, pathogenic effector cells were experimentally induced by immunizing mice with CFA and an antigen expressed in the retina, interphotoreceptor retinoidbinding protein (IRBP). The resulting pathogenic effector cells induce retinal inflammation with clinical features as seen in uveitis. The inflammatory response in the retina is monitored and scored regularly on the basis of severity by examining the fundus.

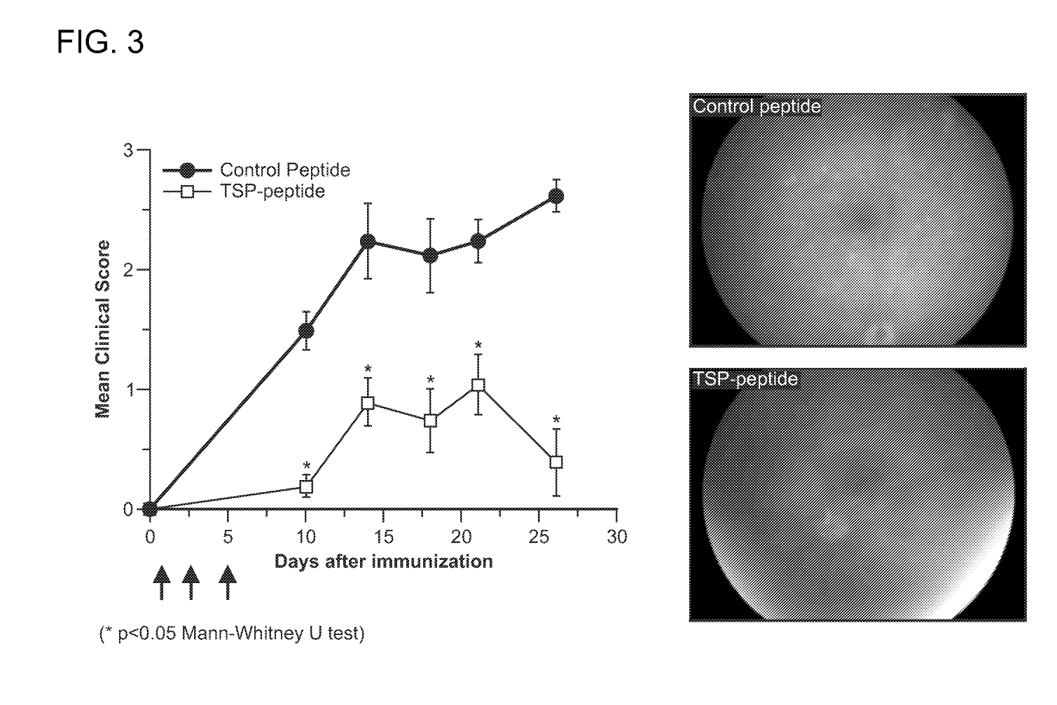

As shown in FIG. 3, disease severity was significantly reduced in mice treated with TSP-1 peptide, as compared to mice treated with control peptide. Representative fundus images are shown as seen on day 26 post-immunization.

C57BL/6 mice were immunized subcutaneously, on day 0, with 200 microgram IRBP in complete Freund's adjuvant per mouse followed by i.p. injection of Pertussis toxin (0.1 microgram). On day 1, 3 and 5 post-immunization mice received i.p. injection of 10 microgram of either control or TSP-peptide. Clinical disease score was recorded for the 26 days post-immunization.

For topical administration, 10 microgram per mouse (5 microgram per eye) was administered for the duration of 7 days. TSP peptide was administered topically once per day for 7 days post-immunization with IRBP-CFA. 1 microgram per microliter solution was used for the eye drops, i.e., 0.1% solution used as 5 microliter per eye resulting in total 10 microliter (10 microgram) per mouse. A liquid formulation was used in topical application as well as when peptide was administered intraperitoneally.

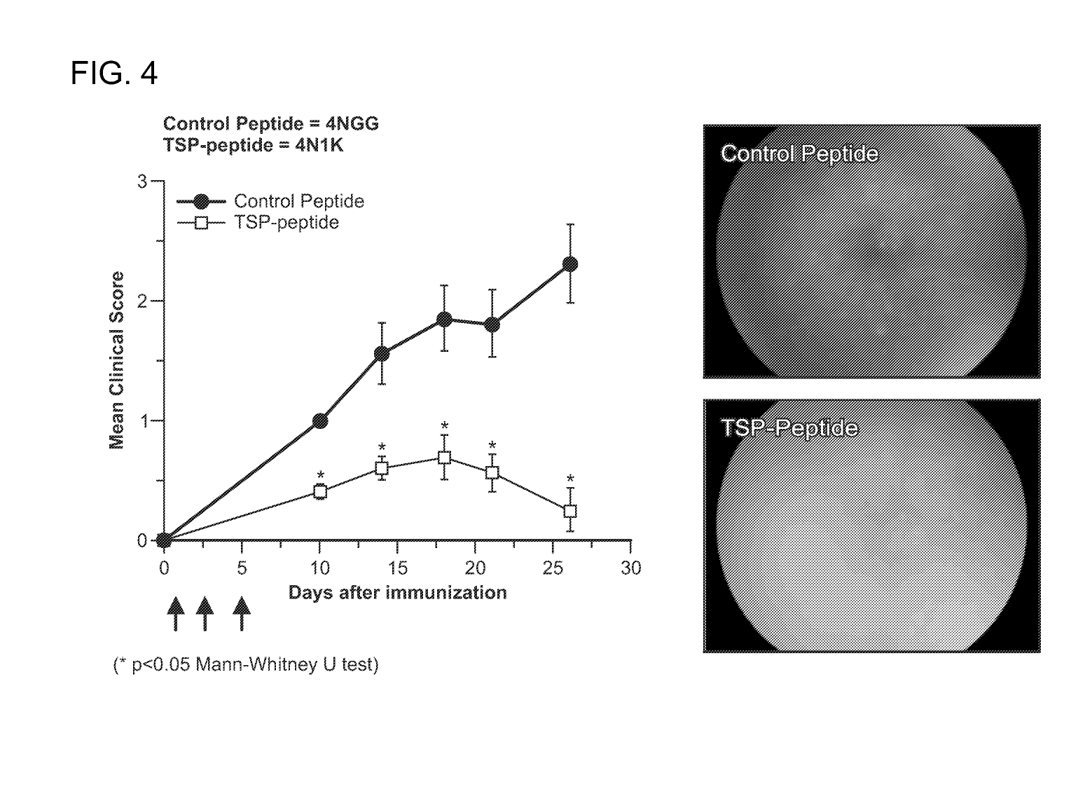

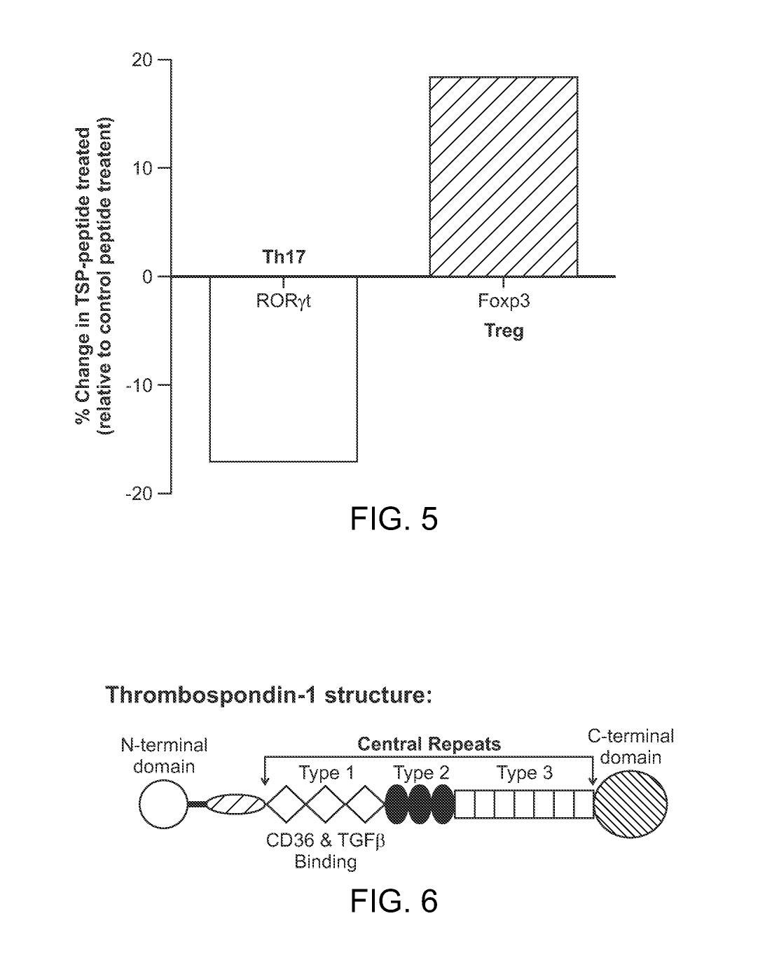

FIG. 4 shows the effect of topical application of TSP-1 derived peptides on the disease course of uveitis in IRBP-immunized mice. FIG. 5 shows the changes in the message for transcriptional factors associated with inflammatory Tₕ17 cells and anti-inflammatory T_{reg} cells in the lymph nodes of TSP-1 peptide-treated mice relative to those treated with the control peptide. These results demonstrate that topical treatment with TSP-1 peptide results in the reduction of ocular inflammation during experimental autoimmune uveitis. Moreover, treatment with TSP-1 peptide was associated with a decline in pro-inflammatory Tₕ17 cells and an enhancement in anti-inflammatory T_{reg} cells in the lymph nodes of treated mice.

C57BL/6 mice were immunized subcutaneously, on day 0, with 200 microgram IRBP in complete Freund's adjuvant per mouse followed by i.p. injection of Pertussis toxin (0.1 microgram). From day 1 to day 7 pos-immunization total 10 microgram of control or TSP-peptide was administered topically (5 micrograms/5 microliters per eye). Clinical disease score was recorded for the 26 days post-immunization.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of increasing a population of anti-inflammatory regulatory T cells (T_{reg}) and decreasing a population of pro-inflammatory T helper 17 (Tₕ17) cells in an ocular or adnexal tissue in a subject, said method comprising topically administering to said ocular or adnexal tissue a composition comprising an effective amount of an agent which binds to a CD47 receptor on T cells, thereby increasing the population of T_{reg} cells and decreasing the population of Tₕ17 cells.

2. The method of claim 1, wherein said agent comprises a C-terminal peptide of thrombospondin-1 (TSP-1) or a fragment thereof.

3. The method of claim 2, wherein said TSP-1 peptide comprises KRFYVVMWKK (SEQ ID NO: 1).

4. The method of claim 1, wherein said population of T_{reg} cells comprises CD4⁺CD25⁺FOXP3⁺ T_{reg} cells which produce transforming growth factor beta (TGF-β).

5. The method of claim 1, wherein said population of Tₕ17 cells produces interleukin-17 (IL-17) or interferon-γ (IFN-γ).

6. The method of claim 1, wherein said composition further comprises a pharmaceutically acceptable carrier.

7. The method of claim 3, wherein said TSP-1 peptide is administered at a dose of 1 μg, 10 μg, 100 μg, or 1,000 μg.

8. The method of claim 1, wherein said composition is present in a concentration of 0.1-10% (mg/ml).

9. The method of claim 1, wherein the form of said composition is a solid, a paste, an ointment, a gel, a liquid, an aerosol, a mist, a polymer, a film, an emulsion, or a suspension.

10. The method of claim 1, wherein said method does not comprise systemic administration or substantial dissemination to non-ocular tissue.

11. The method of claim 1, wherein said composition is incorporated into or coated onto a contact lens.

12. The method of claim 3, wherein said TSP-1 peptide is administered every 48 hours, every 24 hours, every 12 hours, or every 6 hours.

13. The method of claim 3, wherein said TSP-1 peptide is administered for 3 days, 7 days, 14 days, 30 days, 60 days, 90 days, or 120 days.

14. A method for inhibiting or reducing the severity of an inflammatory disorder affecting the ocular and adnexal tissues, comprising topically administering to an ocular or adnexal tissue of a subject a composition that binds to a CD47 receptor on T cells, and increases the population of T_{reg} cells and decreases the population of Tₕ17 cells.

15. The method of claim 14, wherein said composition that binds to a CD47 receptor on T cells comprises a C-terminal peptide of TSP-1 or a fragment thereof.

16. The method of claim 15, wherein said TSP-1 peptide comprises KRFYVVMWKK (SEQ ID NO: 1).

17. The method of claim 11, wherein said inflammatory disorder is an ocular inflammatory disease selected from the group consisting of dry eye disease, uveitis, conjunctivitis, and keratitis.

18. The method of claim 14, wherein said inflammatory disease is not cancer or a tumor.

19. The method of claim 17, further comprising identifying a subject characterized as suffering from an inflammatory disorder affecting the ocular and adnexal tissues.

20. A composition comprising a C-terminal peptide of TSP-1 and a pharmaceutically-acceptable carrier, wherein the form of said composition is a solid, a paste, an ointment, a gel, a liquid, an aerosol, a mist, a polymer, a film, an emulsion, or a suspension.

21. The composition of claim 20, wherein said C-terminal peptide of TSP-1 is about 8, about 9, about 10, or about 11 amino acids in length.

22. The composition of claim 21, wherein said C-terminal peptide of TSP-1 comprises the amino acid sequence KRFYVVMWKK (SEQ ID NO: 1).

23. The composition of claim 20, wherein said composition is incorporated into or coated onto a contact lens.

24. The composition of claim 20, wherein said composition is present in a concentration of 0.1-10% (mg/ml).

25. The composition of claim 20, wherein said pharmaceutically-acceptable carrier is selected from the group consisting of a carbopol gel, a cellulose derivative, detran, gelatin glycerin, polyethylene glycol, poloxamer 407, polysorbate 80, propylene glycol, polyvinyl alcohol, polyvinyl pyrrolidone, and carboxymethylcellulose (CMC).


SEQUENCE LISTING (6 sequences)

SEQ ID NO: 1 - protein, 10 aa, Homo sapiens
KRFYVVMWKK

SEQ ID NO: 2 - DNA, 5760 bp, Homo sapiens
tctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 3 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 4 - protein, 11 aa, Homo sapiens
FIRVVMYEGKK

SEQ ID NO: 5 - protein, 8 aa, Homo sapiens
RFYVVMWK

SEQ ID NO: 6 - protein, 10 aa, Homo sapiens
KRFYVVMWKK